(19) United States
(12) Patent Application Publication
(10) Pub. No.: US 2021/0275409 A1
(43) Pub. Date: Sep. 9, 2021

(54) SKIN CARE COMPOSITION

(71) Applicant: CONOPCO, INC., D/B/A UNILEVER, TRUMBULL, CT (US)

(72) Inventors: WENYAN DONG, Shanghai (CN); Yeyi GU, Shanghai (CN); Lin WANG, Shanghai (CN); Na ZHAO, Shanghai (CN)

(73) Assignee: CONOPCO, INC., D/B/A UNILEVER, TRUMBULL, CT (US)

(21) Appl. No.: 17/258,604

(22) PCT Filed: Jul. 11, 2019
(86) PCT No.: PCT/EP2019/068739

§ 371 Date: Jan. 7, 2021

(30) Foreign Application Priority Data
Aug. 3, 2018 (CN) PCT/CN2018/098520
Sep. 11, 2018 (EP) 18193686.5

(51) Int. Cl. A61K 8/02; A61K 8/25; A61K 8/55; A61K 8/895; A61Q 19/08; A61Q 19/02

(52) U.S. Cl. CPC A61K 8/0279; A61K 8/25; A61K 8/553; A61K 2800/651; A61Q 19/08; A61Q 19/02; A61K 2800/48; A61K 8/895

(57) ABSTRACT

Disclosed is a skin care composition comprising 0.0001 to 10% of phospholipid by weight of the composition, porous particle and silicone elastomer.

### FIELD OF THE INVENTION

The present invention relates to a skin care composition. In particular, the present invention is related to a skin care composition comprising 0.0001 to 10% of phospholipid by weight of the composition, porous particle and silicone elastomer, wherein the weight ratio of the porous particle to the silicone elastomer is from 1:30 to 30:1.

### BACKGROUND OF THE INVENTION

Ageing brings with it many changes to the appearance of skin. Of particular concern to individuals wishing to maintain a youthful appearance is the reduction or elimination of skin imperfections such as wrinkles, age spots or general unevenness of skin tone.

Thus there has been considerable efforts by the cosmetics industry to provide compositions which can mask or at least attenuate skin imperfections. Often this is achieved by creation of a matte effect using materials such as talc, silica, kaolin and other inorganic particulates. These inorganic particulates achieve a matte effect due to their optical properties.

An alternative approach is referred to as achieving blurring effect. Here the incoming light is distorted by scattering (lensing). Components of the cosmetic composition in this mechanism operate as lenses to bend and twist light in a variety of directions.

Traditional approaches, unfortunately, either hide imperfections in the absence of radiance or result in radiance and healthy glow but with aesthetically displeasing skin appearance, for example, through enhanced visibility of skin topography.

International patent application with publication number WO 2017/071886 A1 (Unilever) discloses a personal care composition comprising turbostratic boron nitride, porous silica having a specific surface area of higher than 300 m²/g, and a cosmetically acceptable carrier. Such composition is capable of providing a higher blurring efficacy without compromise of lightness.

We have recognized that there are still needs to improve the blur efficacy. The present inventors developed a skin care composition comprising 0.0001 to 10% of phospholipid by weight of the composition, porous particle and silicone elastomer, wherein the weight ratio of the porous particle to the silicone elastomer is from 1:30 to 30:1. It was surprisingly found that the blur efficacy was significantly improved.

### SUMMARY OF THE INVENTION

In a first aspect, the present invention is directed to a skin care composition comprising 0.0001 to 10% of phospholipid by weight of the composition, porous particle and silicone elastomer, wherein the weight ratio of the porous particle to the silicone elastomer is from 1:30 to 30:1.

In a second aspect, the present invention is directed to a method of reducing the appearance of fine lines, wrinkles, pores and/or blemish spots; evening skin tone, or a combination thereof on skin comprising the step of applying a composition of the present invention on the desired skin surface

In a third aspect, the present invention is directed to use of a composition of the present invention for reducing the appearance of fine lines, wrinkles, pores and/or blemish spots; evening skin tone, or a combination thereof on the desired skin surface.

All other aspects of the present invention will more readily become apparent upon considering the detailed description and examples which follow.

### DETAILED DESCRIPTION OF THE INVENTION

Except in the examples, or where otherwise explicitly indicated, all numbers in this description indicating amounts of material or conditions of reaction, physical properties of materials and/or use may optionally be understood as modified by the word “about”.

All amounts are by weight of the composition, unless otherwise specified.

It should be noted that in specifying any range of values, any particular upper value can be associated with any particular lower value.

For the avoidance of doubt, the word “comprising” is intended to mean “including” but not necessarily “consisting of” or “composed of”. In other words, the listed steps or options need not be exhaustive.

The disclosure of the invention as found herein is to be considered to cover all embodiments as found in the claims as being multiply dependent upon each other irrespective of the fact that claims may be found without multiple dependency or redundancy.

Where a feature is disclosed with respect to a particular aspect of the invention (for example a composition of the invention), such disclosure is also to be considered to apply to any other aspect of the invention (for example a method of the invention) mutatis mutandis.

“Porous particle” as used herein refers to a particle with distributed voids throughout the entire volume of the particle. The voids can be individual or connected by small size openings, similar to pore openings that separate larger spaces.

“Silicone elastomer” as used herein refers to deformable organopolysiloxane with viscoelastic properties.

“Specific surface area” as used herein refers to specific surface area determined according to Brunauer-Emmett-Teller method. The value of the specific surface area was measured by meeting the requirements set out in ASTM standard D 3663-78.

“Diameter” as used herein refers to particle diameter in non-aggregated state unless otherwise stated. For polydisperse samples having particulate with diameter less than 1 μm, diameter means the z-average diameter measured, for example, using dynamic light scattering (see international standard ISO 13321) with an instrument such as a Zetasizer Nano™ (Malvern Instruments Ltd, UK) unless otherwise stated. For polydisperse samples having particulate with diameter no less than 1 μm, diameter means the apparent volume median diameter (D50, also known as x50 or sometimes d(0.5)) of the particles measurable for example, by laser diffraction using a system (such as a Mastersizer™ 2000 available from Malvern Instruments Ltd) meeting the requirements set out in ISO 13320 unless otherwise stated.

Preferably, the phospholipid is derived from lecithin and/or hydrogenated lecithin and more preferably the phospholipid is derived from lecithin. Preferably, the lecithin and/or hydrogenated lecithin is of soy or egg origin.

Preferably, the composition comprises phospholipid in amount of 0.001 to 8% by weight of the composition, more preferably 0.05 to 5%, even more preferably 0.1 to 3% and still even more preferably from 0.2 to 1.5% by weight of the composition.

Preferably, the composition comprises a) lecithin and/or hydrogenated lecithin; (b) porous particle; and (c) silicone elastomer, wherein the total amount of the lecithin and hydrogenated lecithin is 0.0001 to 10% by weight of the composition. More preferably, the total amount of the lecithin and hydrogenated lecithin more preferably 0.05 to 7%, even more preferably 0.1 to 4% and still even more preferably from 0.2 to 2% by weight of the composition.

The requirement for porous particle of the present invention is that the porous particle has the capability of absorbing large amounts of oils. Preferably, the porous particle has an oil absorption value of higher than 50 g/100 g, more preferably higher than 200 g/100 g, and even more preferably higher than 300 g/100 g. The oil absorption value is preferably in the range of 50 g/100 g to 1500 g/100 g, more preferably in the range of 200 g/100 g to 1200 g/100 g and even more preferably in the range of 300 g/100 g to 1100 g/100 g. The oil absorption value refers to the values measured in conformity with ASTM Method D281-84.

The specific surface area of the porous particle is preferably at least 100 m²/g, more preferably from 200 to 1000 m²/g, even more preferably from 550 to 880 m²/g and most preferably from 590 to 810 m²/g.

The porous particle of the invention has high pore volume to ensure the capability of oil absorption. Preferably, the pore volume value of the porous particle is higher than 0.02 cc/g, more preferably higher than 0.7 cc/g, and even more preferably higher than 1.5 cc/g. The pore volume value is preferably in the range of 0.02 cc/g to 7 cc/g, more preferably in the range of 0.7 cc/g to 6 cc/g and even more preferably in the range of 1.5 cc/g to 5.6 cc/g. The pore volume value refers to the values measured in conformity with ASTM D4222-03.

The porous particle preferably has an average diameter of 200 nm to 50 microns, more preferably from 500 nm to 10 microns, and most preferably from 1 to 6 microns. To have a better sensory, the porous particle is preferably substantially uniform in size which means less than 5% of the porous particle have a diameter less than 0.5 times the average diameter and less than 5% of the porous particle have a diameter greater than 1.5 times the average diameter. In another aspect, the range of the diameter of the porous particle is preferably 0.8 to 1.2 times the average diameter, more preferably 0.9 to 1.1 times the average diameter.

Preferably, the porous particle is hydrophilic. The porous particle preferably comprises silica, cellulose, starch, acrylic polymer, nylon, or a mixture thereof. The acrylic polymer means polymer comprising, as polymerized monomer, acrylic acid, its homologues, and/or derivatives thereof; preferably acrylic acid, methacrylic acid, esters of acrylic acid, esters of methacrylic acid, combination thereof, or derivative thereof. More preferably, the porous particle comprises silica, nylon, polymer containing methacrylate and/or acrylate as polymerized monomer, or a mixture thereof, Even more preferably, the porous silica is selected from silica, nylon, or a mixture thereof. Most preferably, the porous particle is porous silica. Commercially available porous silica which may be used in the present invention are MSS-500/3H and MSS-500/H from Kobo.

Preferably, the porous particle is present in the composition in amount of from 0.01 to 10% by weight of the composition, more preferably 0.1 to 7%, even more preferably from 0.3 to 4%, and still even more preferably from 0.5 to 2% by weight of the composition.

The silicone elastomer used in the present invention is preferably powder of silicone elastomer. The silicone elastomer is different from the porous particle.

It is highly preferred that the silicone elastomer is cross-linked. The silicone elastomer can be obtained from curable organo-polysiloxanes. Examples in this respect are:

addition reaction-curing organopolysiloxane compositions which cure under platinum metal catalysis by the addition reaction between SiH-containing diorganopolysiloxane and organopolysiloxane having silicon-bonded vinyl groups; condensation-curing organopolysiloxane compositions which cure in the presence of an organotin compound by a dehydrogenation reaction between hydroxyl terminated diorganopolysiloxane and

SiH-containing diorganopolysiloxane; condensation-curing organopolysiloxane compositions which cure in the presence of an organotin compound or a titanate ester, by a condensation reaction between a hydroxyl terminated diorganopolysiloxane and a hydrolyzable organosilane (this condensation reaction is exemplified by dehydration, alcohol-liberating, oxime-liberating, amine-liberating, amide-liberating, carboxyl-liberating, and ketone-liberating reactions); peroxide-curing organopolysiloxane compositions which thermally cure in the presence of an organoperoxide catalyst; and organopolysiloxane compositions which are cured by high-energy radiation, such as by gamma-rays, ultraviolet radiation or electron beams. The silicone elastomer is preferably obtained by addition reaction-curing organopolysiloxane compositions which cure under platinum metal catalysis by the addition reaction between SiH-containing diorganopolysiloxane and organopolysiloxane having silicon-bonded vinyl groups

The silicone elastomer may either be an emulsifying or non-emulsifying cross-linked silicone elastomer or a combination thereof but preferably the silicone elastomer is non-emulsifying. The term “non-emulsifying,” as used herein, defines cross-linked silicone elastomer from which poly-oxyalkylene units are absent. The term “emulsifying,” as used herein, means cross-linked organo-polysiloxane elastomer having at least one poly-oxyalkylene (e.g., poly-oxyethylene or poly-oxypropylene) unit.

Preferred silicone elastomers are organo-polysiloxanes available under the INCI names of dimethicone/vinyl dimethicone crosspolymer, dimethicone crosspolymer and Polysilicone-11. More preferably the silicone elastomer is dimethicone/vinyl dimethicone crosspolymer.

Typically, the average diameter of the silicone elastomer is from 0.2 to 50 microns, more preferably from 0.5 to 20 microns, even more preferably from 0.8 to 10 microns, and still even more preferably from 1.5 to 6 microns.

The silicone elastomer is preferably present in amount of 0.001 to 15%, more preferably 0.05 to 8%, even more preferably from 0.3 to 3%, still even more preferably from 0.5 to 2% by weight of the composition.

To further improve the and/or blur efficiency and/or the sensory of the composition, preferably the weight ratio of the phospholipid to the porous particle is from 1:60 to 20:1, more preferably 1:25 to 8:1, even more preferably from 1:10 to 2:1 and most preferably 1:4 to 1.5:1. Preferably, the weight ratio of the phospholipid to the silicone elastomer is from 1:60 to 20:1, more preferably 1:25 to 8:1, even more preferably from 1:10 to 2:1 and most preferably 1:4 to 1.5:1. The weight ratio of the porous particle to the silicone elastomer is from 1:30 to 30:1, preferably 1:15 to 15:1, more preferably from 1:6 to 6:1 and most preferably 1:3 to 3:1.

To further improve the and/or blur efficiency and/or the sensory of the composition, preferably the weight ratio of the total amount of lecithin and hydrogenated lecithin to the porous particle is from 1:60 to 20:1, more preferably 1:25 to 8:1, even more preferably from 1:10 to 2:1 and most preferably 1:4 to 1.5:1. Preferably, the weight ratio of the total lecithin and hydrogenated to the silicone elastomer is from 1:60 to 20:1, more preferably 1:25 to 8:1, even more preferably from 1:10 to 2:1 and most preferably 1:4 to 1.5:1.

Preferably, the composition additionally comprises an aerogel particle. The aerogel particle is different from the porous particle. The aerogel particle is also different from the silicone elastomer. Preferably, the weight ratio of the diameter of the aerogel particle to the diameter of the porous diameter is 1:1 to 10:1, and more preferably 1.2:1 to 6:1.

Typically, the aerogel particle comprises polyurethanes, polyacrylates, polymethacrylates, polyolefins, polystyrenes, polyvinyl alcohol, celluloses, celluloses derivatives, chitosan, starch, silica, or a mixture thereof. Preferably, the aerogel particle comprises inorganic material. More preferably the aerogel particle comprises silica. Even more preferably the aerogel particle comprises silylated silica and most preferably the aerogel particle has a INCI name of silica silylate.

The specific surface area of the aerogel particle is preferably at least 100 m²/g, more preferably from 200 to 1500 m²/g, even more preferably from 500 to 1000 m²/g and most preferably from 590 to 810 m²/g. The aerogel particle preferably has an average diameter of 1 to 70 microns, more preferably from 3 to 25 microns, and most preferably from 5 to 15 microns.

Preferably, the aerogel particle is present in the composition in amount of from 0.01 to 10% by weight of the composition, more preferably 0.1 to 7%, even more preferably from 0.3 to 4%, and still even more preferably from 0.5 to 2% by weight of the composition.

Preferably the composition comprises a thickening agent. A variety of thickening agents may be included in the compositions. Illustrative but not limiting are Acrylamide/Sodium Acryloyldimethyltaurate Copolymer (Aristoflex AVC), Hydroxyethyl Acrylate/Sodium Acryloyldimethyltaurate Copolymer, Aluminum Starch Octenyl Succinate, Polyacrylates (such as Carbomers including Carbopol® 980, Carbopol® 1342, Pemulen TR-2® and the Ultrez® thickeners), Polysaccharides (including xanthan gum, guar gum, pectin, carageenan and sclerotium gums), celluloses (including carboxymethyl cellulose, ethyl cellulose, hydroxyethyl cellulose and methyl hydroxymethyl cellulose), minerals (including talc, silica, alumina, mica and clays, the latter being represented by bentonites, hectorites and attapulgites), magnesium aluminum silicate and mixtures thereof. Preferably, the thickening agent is selected from Carbomer, taurate copolymer, acrylate copolymer or a mixture thereof. More preferably, the thickening agent is acrylate copolymer.

Amounts of the thickening agent may range, for example, from 0.05 to 10%, more preferably from 0.1 to 5%, even more preferably from 0.3 to 2% by weight of the composition.

Compositions of the present invention may include a cosmetically acceptable carrier. The carrier is preferably a liquid. Water is the most common carrier. The composition is preferably an emulsion and more preferably and oil-in-water emulsion.

The composition may comprise water in amount of 35 to 95% by weight of the composition, more preferably from 45 to 92%, even more preferably from 50 to 90%, most preferably from 68% to 88% by weight of the composition.

The composition may comprise optional ingredients including whitening pigment, emollient material, moisturizing agent, organic sunscreen, skin lightening agent, fragrance, natural extract, or a combination thereof.

Whitening pigments are typically particles of high refractive index materials. For example, the whitening pigment may have a refractive index of greater than 1.3, more preferably greater than 1.8 and most preferably from 2.0 to 2.7. Examples of such whitening pigment are those comprising bismuth oxy-chloride, boron nitride, barium sulfate, mica, silica, titanium dioxide, zirconium oxide, aluminium oxide, zinc oxide or combinations thereof. More preferred whitening pigment are particles comprising titanium dioxide, zinc oxide, zirconium oxide, mica, iron oxide or a combination thereof. Even more preferred whitening pigment are particles comprising zinc oxide, zirconium oxide, titanium dioxide or a combination thereof as these materials have especially high refractive index. Still even more preferably the whitening pigment is selected from titanium dioxide, zinc oxide or a mixture thereof and most preferred whitening pigment is titanium dioxide. The average diameter of whitening pigment is typical from 15 nm to 1 microns, more preferably from 35 nm to 800 nm, even more preferably from 50 nm to 500 nm and still even more preferably from 100 to 300 nm.

Suitable emollient materials include silicones, hydrocarbons, triglycerides or a mixture thereof. These silicones may be organic, silicone-containing or fluorine-containing, volatile or non-volatile, polar or non-polar. Hydrocarbons may include mineral oil, petrolatum and polyalpha-olefins. Examples of preferred volatile hydrocarbons include polydecanes such as isododecane and isodecane (e.g. Permethyl-99A which is available from Presperse Inc.) and the C7-C8 through C12-C15 isoparaffins (such as the Isopar Series available from Exxon Chemicals). Illustrative triglycerides but not limiting are sunflower seed oil, cotton oil, canola oil, soybean oil, castor oil, borage oil, olive oil, shea butter, jojoba oil and mixtures thereof. Mono- and di-glycerides may also be useful. Particularly preferable are glyceryl monostearate and glyceryl distearate.

Particularly preferred moisturizing agents includes, petrolatum, aquaporin manipulating actives, oat kernel flour, substituted urea like hydroxyethyl urea, hyaluronic acid and/or its precursor N-acetyl glucosamine, hyaluronic acid and/or its precursor N-acetyl glucosamine, or a mixture thereof.

A wide variety of organic sunscreen is suitable for use in combination with the essential ingredients of this invention. Suitable UV-A/UV-B sunscreen include, 2-hydroxy-4-methoxybenzophenone, octyldimethyl p-aminobenzoic acid, digalloyltrioleate, 2,2-dihydroxy-4-methoxybenzophenone, ethyl-4-(bis(hydroxypropyl)) aminobenzoate, 2-ethylhexyl-2-cyano-3,3-diphenylacrylate, 2-ethylhexylsalicylate, glyceryl p-aminobenzoate, 3,3,5-trimethylcyclohexylsalicylate, methylanthranilate, p-dimethyl-aminobenzoic acid or aminobenzoate, 2-ethylhexyl-p-dimethyl-amino-benzoate, 2-phenylbenzimidazole-5-sulfonic acid, 2-(p-dimethylaminophenyl)-5-sulfonicbenzoxazoic acid, 2-ethylhexyl-p-methoxycinnamate, butylmethoxydibenzoylmethane, 2-hydroxy-4-methoxybenzophenone, octyldimethyl-p-aminobenzoic acid and mixtures thereof. The most suitable organic sunscreens are 2-ethylhexyl-p-methoxycinnamate, butylmethoxydibenzoylmethane or a mixture thereof.

Vitamin B3 compounds (including derivatives of vitamin B3) e.g. niacin, nicotinic acid or niacinamide are the preferred skin lightening agent as per the invention, most preferred being niacinamide.

The compositions of the present invention can comprise a wide range of other optional components. Examples include antioxidants, colorants, fragrance, and preservatives.

Preferably, the composition has a L&W (line and wrinkle) index of at least −50%. More preferably the composition has a L&W index of 0% to 300%. Even more preferably, the skin care composition has a L&W index of 25% to 200%. The measurements of L&W index and lightness are described in Example 2.

The skin care composition refers to a composition suitable for topical application to human skin, preferably is a leave-on product. The term “leave-on” as used with reference to compositions herein means a composition that is applied to or rubbed on the skin, and left thereon. The term “skin” as used herein includes the skin on the face (except eye lids and lips), neck, chest, abdomen, back, arms, under arms, hands, and legs. Preferably “skin” means includes the skin on the face (except eye lids and lips) and under arms, more preferably skin means skin on the face other than lips and eyelids.

The following examples are provided to facilitate an understanding of the invention. The examples are not intended to limit the scope of the claims.

### EXAMPLES

Material

TABLE 1

| | | | Diameter |
|---|---|---|---|
| Trade name | INCI name | Supplier | (microns) |
| EMULMETIK 300 | Lecithin | LUCASMETER | — |
| MSS-500/3H* | Silica (Porous) | KOBO | 3 |
| DC9509 (63% | Dimethicone/Vinyl | DOW CORNING | 3 |
| solid active) | dimethicone | | |
| | Crosspolymer (and) | | |
| | C12-14 Pareth-12 | | |
| VM-2270 | Silica silylate | DOW CORNING | 5-15 |
| Luvigel EM | Sodium Acrylates | BASF | — |
| | Copolymer | | |

*The values of surface area and oil absorption are 600-800 m²/g and 300 g/100 g respectively.

### Example 1

TABLE 2

Samples (wt %)*

| Ingredient | A | B | 1 |
|---|---|---|---|
| Tween 20 | — | 1.00 | — |
| Lecithin | 1.00 | — | 1.00 |
| Porous silica | — | 1.00 | 1.00 |
| Dimethicone/Vinyl dimethicone | — | 1.00 | 1.00 |
| Crosspolymer | | | |
| Silica silylate | 1.00 | 1.00 | 1.00 |
| Disodium EDTA | 0.10 | 0.10 | 0.10 |
| Sodium Acrylates Copolymer | 2.50 | 2.50 | 2.50 |
| Caprylic/Capric Triglyceride | 3.13 | 3.13 | 3.13 |
| Dicaprylyl Carbonate | 3.00 | 3.00 | 3.00 |
| Shea Butter | 1.00 | 1.00 | 1.00 |
| Preservative | 0.45 | 0.45 | 0.45 |
| Water | to 100 | to 100 | to 100 |

*The level of the ingredients refers the level of active.

A series of skin care compositions were formulated as shown in Table 2 by using materials in Table 1.

### Example 2

This example demonstrated that the improved instant blurring efficiency of the skin care composition of the present invention.

(1). Measurement of the gloss degree of the artificial skin before and after the skin care compositions was applied.

Wrinkled Bio-skin plates (BP-EW1 #BSC, Beaulax Co., Ltd., Tokyo, Japan) made of polyurethane elastomer were used as substrate to mimic the human skin with wrinkles. A dual-polarized image system called SAMBA (Bossa Nova Technologies, USA) was employed to measure the gloss degree of the wrinkled Bio-skin plates by following the method and principle described by Akira Matsubara [Skin translucency: what is it and how is it measured, The International Federation of Societies of Cosmetic Chemists (IFSCC) Congress 2006, Osaka, Japan]. A software named SAMBA face system (Version 4.3) was equipped for the analysis. The Wrinkled Bio-skin plates were tested against an incident light with exposure time of 80 msec. The operation mode was parallel polarization and crossed polarization modes.

Then, 28 mg of one sample as prepared in Example 1 was applied to and spread by finger cot within the circle with area of 7 cm² for gloss test and wait for 30 minutes to dry naturally. The gloss of the wrinkled Bio skin plates after the samples were applied were measured again using the SAMBA system.

(2). Calculation of L&W Index

The incident light was reflected and scattered by Bio-skin plates. The specular reflected light kept the same polarization as the incident light whereas the scattering light from the volume (diffused light) was un-polarized. The SAMBA camera acquired successively two images corresponding to two states of polarization (parallel and crossed). The parallel image intensity (P) is contributed from both the reflected and scattered light, and the crossed image intensity (C) is contributed from the scattered light only. The parallel image plus the crossed image is equal to the total image delivered by a traditional camera or perceived by human eye.

The gloss degree was calculated by (P−C)/(P+C). The calculation of gloss degree was performed for each pixel. The standard deviation (STD) of gloss degree is a measure of the uniformity of the skin appearance. The higher the STD is, the lower the uniformity is. Herein we defined a L&W (line and wrinkle) index to demonstrate degree of blurring efficacy of the skin care composition. The L&W index was calculated by (STD of gloss degree before applying sample—STD of gloss degree after applying sample)/(STD of gloss degree before applying sample). The higher the L&W index is, the higher is the blurring efficacy of the sample.

TABLE 3

| Sample | L&W index |
|---|---|
| A | −58.6% ± 2.4% |
| B | 26.9% ± 1.5% |
| 1 | 35.5% ± 1.1% |

The data of blurring efficacy is listed in Table 3. The data indicates that the composition according to the present invention provided better instant blurring efficacy than composition containing tween 20 (Sample 1 vs. Sample B). It was surprisingly found that the phospholipid is capable of boosting the instant blurring efficacy of the skin care composition containing porous particle and silicone elastomer.

1. A skin care composition comprising:
(a) 0.0001 to 10% of phospholipid by weight of the composition;
(b) porous particle; and
(c) silicone elastomer,
wherein the weight ratio of the porous particle to the silicone elastomer is from 1:30 to 30:1 and the silicone elastomer is different from the porous particle.

2. The composition according to claim 1, wherein the phospholipid is derived from lecithin and/or hydrogenated lecithin.

3. The composition according to claim 1, wherein the amount of the phospholipid is 0.001 to 8% by weight of the composition.

4. The composition according to claim 1, wherein the porous particle has an average diameter of from 200 nm to 40 microns.

5. The composition according to claim 1, wherein the porous particle is porous silica.

6. The composition according to claim 1, wherein the porous particle is present in amount of 0.01 to 10% by weight of the composition.

7. The composition according to claim 1, wherein the weight ratio of the phospholipid to the porous particle is from 1:25 to 8:1.

8. The composition according to claim 1, wherein the weight ratio of the phospholipid to the porous particle is from 1:4 to 1.5:1.

9. The composition according to claim 1, wherein the silicone elastomer is a vinyl dimethicone/dimethicone crosspolymer and the amount of the silicone elastomer is 0.05 to 8% by weight of the composition.

10. The composition according to claim 1, wherein the weight ratio of the porous particle to the silicone elastomer is from 1:6 to 6:1.

11. The composition according to claim 1 additionally comprising aerogel particle.

12. The composition according to claim 11, wherein the aerogel particle is present in the composition in amount of from 0.01 to 10% by weight of the composition.

13. The composition according claim 1, wherein the composition comprises a thickening agent.

14. A method of reducing the appearance of fine lines, wrinkles, pores and/or blemish spots; evening skin tone, or a combination thereof on skin comprising the step of applying a composition according to claim 1 on the desired skin surface.

15. The composition according to claim 1, wherein the phospholipid is derived from lecithin and the amount of the phospholipid is 0.1 to 3% by weight of the composition.

16. The composition according to claim 1, wherein the lecithin and/or hydrogenated lecithin is of soy or egg origin.

17. The composition according to claim 1, wherein the porous particle has an average diameter of from 1 to 20 microns and is present in amount of 0.1 to 7% by weight of the composition.

18. The composition according to claim 1, wherein the aerogel particle is present in the composition in amount of from 0.3 to 4% by weight of the composition and further wherein the aerogel particle is silica silylate.

19. The composition according to claim 13, wherein the thickening agent is acrylate copolymer.

20. The composition according to claim 1, wherein the amount of the silicone elastomer is 0.5 to 2% by weight of the composition.
